(19) United States
(12) Patent Application Publication
(10) Pub. No.: US 2003/0138868 A1
(43) Pub. Date: Jul. 24, 2003

(54) Agents and method for diagnosing lyme disease and borreliosis vaccine

(76) Inventors: Peter Jungblut, Berlin (DE); Aydan DIlgimen, Berlin (DE); Sascha Thies, Berlin (DE); Brigitte Wittmann, Berlin (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(21) Appl. No.: 10/149,532

(22) PCT Filed: Dec. 8, 2000
(86) PCT No.: PCT/EP00/12454

(30) Foreign Application Priority Data
Dec. 8, 1999 (DE) 19960113.5

(51) Int. Cl. C12Q 1/68; G01N 33/554; G01N 33/569; C12Q 1/48

(52) U.S. Cl. 435/7.32; 435/15; 435/6

(57) ABSTRACT

The invention relates to agents for diagnosing and/or treating Lyme disease which contain the antigens of glyceraldehyde-3-phosphate-dehydrogenase (GAPDH), oligopeptide permease, oligopeptide ABC transporter periplasmic BP(oppA-2)(Bb), glycosyl transferase IgtD homologue, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane assoc. protein p66 percursor Borrelia burgdoferii, oligopeptide ABC transporter periplasmic BP (oppA-4)(Bc), fructose-biphosphate aldose (fba) Borrelia burgdorferi, DNAK protein Heat Shock Protein 70 Borrelia burgdorferi, orfE Borrelia burgdorferi, outer surface protein B precursor Borrelia burgdorferi, L-lactate dehydrogenase (ldh), P83/100 gene Borrelia burgdorferi, enolase 2-phosphoglycerate Borrelia burgdorferi, flagellin Protein Borrelia garinii, hypothetical protein BBE28 Borrelia burgdorferi, DNA direct. RNA polymerase (rpoA) homolog, P66 protein (fragment), flagellin (fragment), DNA direct. RNA polymerase, integral outer membran protein p66, pyruvate kinase (pyk) homolog, phosphoglycerate kinase (pgk) and/or BBU28760 NID, and/or fragments thereof and/or the nucleic acid sequences encoding said antigens and/or said fragments.

The invention relates to agents and a method for the diagnosis of Lyme Disease and a Borreliosis-vaccine. The following antigens of Borreliosis were identified, which are characterized by high specifity and sensitivity: Glyceraldehyde-3-phosphate-dehydrogenase GAPDH, oligopeptide permease, oligopeptide ABC transporter periplasmic BP (oppA-2)(Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane assoc. protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (ldh), P83/100 gene Borrelia burgdorferi, enolase 2-phosphoglycerat Borrelia burgdorferi, flagellin protein Borrelia garinii, hypothetical protein BBE28 Borrelia burgdorferi, DNA direct. RNA polymerase (rpoA) homolog, P66 protein (fragment), flagellin (fragment), DNA direct. RNA polymerase, integral outer membran protein p66, pyruvate kinase (pyk) homolog, phosphoglycerat kinase (pgk) and/or BBU28760 NID.

One of the most common human infectious diseases transferred by tick-borne bacteria is Lyme Borreliosis (LM). The illness is caused by Spirochetes of Borrelia burgdorferi complexes. The illness is manifested in different variations: e.g., as Erythema migrans (EM), Neuroborreliosis, Akrodermatitis chronica athrophicans (ACA), Lymphadenosis cutis benigna (LCB), Lymphozytic Meningoradiculitis (Bannwarth-Syndrome).

The species of Borrelia burgdorferi complexes (also named Borrelia burgdorferi sensu lato) are divided in three main genospecies, B. burgdorferi sensu stricto, B. garinii und B. afzelii.

Balmelli, T. and Piffaretti, J. C. (Int. J. Syst. Bacteriol. 1996, 46, 167-172) as well as Valsangiacomo, C. et al. (Int. Syst. Bacteriol. 1997, 47, 1-10) have further subdivided in five species.

While B. burgdorferi sensu stricto appears mainly in Northamerica, B. garinii is common in Europe, and in Asia B. afzelii is most common. Sera of Lyme Borreliosis patients usually contain only antibodies against one of the three most known species; however, these may crossreact.

With the immunogenic protein OspA (outer-surface lipoprotein), a known test antigen of Borreliosis, an appropriate vaccine could be made available. This polyvalent vaccine OspA protects mice against B. burgdorferi sensu stricto, B. garinii und B. afzelii (Gern, L. Vaccine 1997, 15 1551-1557, compare also attachment 1).

Since significant differences of OspA exist in the various Borrelia species, especially apart from Northamerica, it is only conditionally suitable as a test antigen and for the use in diagnosis kits.

Hence, still multiple vaccines are mandatory for the maily in Europe and in Asia occurring B. burgdorferi, B. garinii und B. afzelii which are directed against the European and Asiatic Borrelia species. A vaccine therefore must contain different or further proteins and/or peptides, in order to provide a broad protection.

Objective of the invention was to find further antigens for the diagnosis of LM and to provide a suitable vaccine, which allows for a broad application.

Most surprisingly with the identification of the glycolytic enzyme glyceraldehyde-3-phosphate-dehydrogenase (GAPDH), oligopeptide permease, oligopeptide ABC transporter periplasmic BP(oppA-2)(Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane associated protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 90 and/or 70 Borrelia burgdorferi, orfE Borrelia burgdorferi, outer surface protein B precursor Borrelia burgdorferi, 1-lactate dehydrogenase (LDH), P83/100 gene Borrelia burgdorferi, enolase 2-phosphoglycerate Borrelia burgdorferi, flagellin protein Borrelia garinii, hypothetical protein BBE28 Borrelia burgdorferi, DNA directed RNA polymerase (rpoA) homolog, P66 protein (fragment), flagellin (fragment), DNA directed RNA polymerase, integral outer membran protein p66, pyruvate kinase (pyk) homolog, phosphoglycerate kinase (pgk) and/or BBU28760 NID antigens were found, which can successfully be used as proof of Borreliosis in early appearance as well as also in late stage of disease.

In the context of the presented invention the following general terms shall be used as follows:

In the context of the presented invention the term pharmaceutical or diagnostic composition means a substance which is suitable for the diagnosis and/or treatment of diseases, especially of those illnesses in context with Borreliosis, preferably in an amount sufficient for reaching such effect. The term “pharmaceutical or diagnostical composition” employed in the presented description means the active substance as well as the active substance in combination with pharmaceutical supports, adjuvants, or other active substances.

In the context of the presented invention the term treatment means the prophylactic and/or therapeutical effect of a pharmaceutical substance.

In the context of the presented invention the term Borreliosis antigen concerns the natural occurring Borreliosis antigens as well as all modifications, mutants or derivatives of the Borreliosis antigens, antigens produced by recombinant techniques, the amino acid modifications such as inversions, deletions, insertions, extensions, etc., as far as the essential functions are concerned of the wild-type Borreliosis antigens. Such Borreliosis antigens can also contain unusual amino acids, and/or modifications, such as alkylation, oxidation, thiol-modification, denaturation and oligomerization and such.In the context of the presented invention the term Borreliosis antigens can in particular mean proteins and/or peptides and/or fusion peptides, which contain complete or parts of Borreliosis antigens together with other proteins, peptides or parts thereof. In a further form of the presented invention the Borreliosis antigens are shortened parts of the naturally occurrring Boreliosis antigens, auch as small peptides.

The term promoter means a DNA-sequence, which usually is upstream (5′) of the coding sequence of a structural gene and which controlles the expression of the coding region, especially of the Borreliose antigen, by providing a recognition sequence of the RNA-Polymerase and/or of other factors which are necessary for the correct start of the transcription. Promoter sequences are necessary, however, not always sufficient to govern the expression of the gene.

In the sense of the invention nucleic acid means a big molecule, which can be single- or double-stranded and consists of monomers (nucleotides) consisting of a carbohydrate unit, a phosphate unit and either one of a purine or pyrimidine residue. The nucleic acid may be cDNA, genomic DNA or RNA, for example mRNA.

The term nucleic acid sequence means a naturally occurring or synthetized polymer of single- or double stranded DNA or RNA, which alternatively contain synthetic, non-natural or altered nucleotide bases which could be built in DNA- or RNA polymers.

The term gene means a specific protein coding for a DNA-sequence and regulation element, which controls the expression of this DNA-sequence.

According to the invention the term coding sequence apply to the part of a gene, which codes for a protein, a polypeptide or part thereof whereby the regulation sequences and/or -elements are excluded, which govern the initiation or termination of the transcription. The coding sequence and/or the regulation element can be one typically found in the cell, in this case termed autologous or endogenous, or one, usually not localized in the cell whereby in this case termed heterologous.

A heterologous gene may consist of autologous elements arranged in a sequence and/or orientation which typically is not found in the cell to which the gene is transferred to. A heterologous gene may derive in total or in part from any given source in the field to which a bacterial or viral genome or episome, eukaryotic nucleus or plasmid-DNA, cDNA or chemically synthetized DNA count. The structural gene may form a non-interrupted coding region or may contain one or more introns which by splicing elements form the boundary. The structural gene may consist of naturally occurring areas or derive from synthetic origin.

A gene product proven is a nucleotide- or amino acid sequence, especially a Borreliosis antigen which may be proven by the aid of a test. Preferably the expression of a proven gene of a cell marks a feature, allowing for a simple selection of a cell from others not expressing the proven gene product.

The term vector means a recombinant DNA construct, which may be a plasmide, a virus or an autonomous replicating sequence, a phage or a linear or circular nucleotide, consisting of a single- or double stranded DNA or RNA, which are connected or recombined by a sequence of nucleotide sequences to a unique construct and which can insert a promoter fragment and a DNA sequence of a selected gene product in sense- oder antisense orientation in combination with a suitable non-translated 3′-sequence into a cell.

Plasmides are genetic elements, which can be transmitted in stable form, without being part of the chromosome of the host cell. They may comprise linear or circular DNA or RNA. Plasmides code for molecules, which during cell replication secure their replication and the stable heredity, and may be products of considerable value for medicine, agriculture and environment. For example they code for toxines which enlarge the virulence of pathogenic bacteria. They may also code for resistance against antibiotica. Plasmides are typically used in molecular biology as vectors for cloning and expression of rekombinant genes. Corresponding to the standard expert rules plamides are marked by a small letter p to which large case letters and/or numbers are added in front or thereafter. In the present invention the disclosed starting plasmides are either commenly available, open to the public or may bIe constructed from commenly available plasmides to the public or may be constructed from available plasmides by routine application of common routine processes. Many plasmides and other cloning and expression vectors which may be used in the invention are wellknown and available to the experts. Furthermore, the expert may construct without special skill any number of other plasmides which are suitable for the invention. By the invention it is evident that the expert is able to get the properties, the construction and application of such plasmides as well as of other vectors without special skill.

The term expression in the present application describes the transcription and/or coding of the sequence of the gene product, for instance of Borreliosis antigens. At the expression firstly a DNA-chain coding for the sequence of a gene product is transcribed into a complementary RNA, most usually a mRNA, and then the transcribed mRNA is translated into the above mentioned gene product if this codes for a protein. The expression also implies the transcription of DNA, which according to its regulation elements is inserted in antisense direction. The expression can also comprise overexpression of a gene product in a constitutive process most possibly further enhanced through a promoter fragment controlled from the outside whereby several mRNA-copies and large amounts of the selected gene product are produced.

The invented peptides or proteins, especially of the Borreliose antigens and proteins, respectively, were isolated and characterized in purified form. In the present invention the term isolated means that the proteins/polypeptides are produced without any material or at least with part thereof with which they are in contact in their natural environment. On the basis of the molecular mass of the total protein in a certain probe the isolated protein is contained to minimal 0.5%, preferably minimal 5%, favourably minimal 25% and more favourably to minimal 50%. Most favoured is the isolated protein mostly freed of any other protein, lipids, carbohydrates and- other substances, with whom is it associated under natural conditions, and that it forms a single band on a polyacrylamide gel, and a single protein spot on a two-dimensional gel. Essential free means, that the protein is contained to a minimum of 75%, preferably to a minimum of 85%, favourably to al least 95% und most favourable to at least 99% freed of other proteins, lipids, carbohydrates and other substances with whom it is associated under natural conditions.

Antibody means a polypeptide, which essentially is coded for by an immunoglobulin gene or immunoglobin gene or fragments thereof which binds and recognizes specifically an analyte (antigen). Known immunoglobin genes comprise the kappa-, lambda-, alpha-, gamma-, delta-, epsilon- und mu-genes for the constant region as well as the innumerous genes for the variable immunoglobulin region. Antibodies for instance exist as intat immunoglobulines or as several well characterized fragments, produced by cleavage with different proteases. The term antibodies means also modified antibodies (e.g. oligomers, reduced, oxidised and labeled antibodies). The term antibody used in this invention comprises also antibody fragments, produced either by modification of intact antibodies or obtained by de-novo synthesis under application of DNA-rekombinant techniques. The term antibody encloses intact molecules as well as fragments thereof, such as Fab, F(ab′)₂ and Fv, which may bind the epitope binding determinants. These fragments kept in part the ability of the antibody to selectively binding of its antigen or receptor whereby the definition is:

(1) Fab, the fragment which contains a monovalent antigen-binding fragment of an antibody molecule, can be obtained by cleavage of an entire antibody by the enzyme papain, whereby an intact light chain and a part of a heavy chain are obtained;

(2) the Fab′-fragment of an antibody molecule is obtainable by pepsin treatment and further reduction of an entire antibody whereby an intact light chain and a part of a heavy chain are obtained; two Fab′-fragments are obtained by one antibody molecule;

(3) F(ab′)₂, the fragment of the antibody, which is obtained by treatment of the entire antibody by the enzyme pepsin without any reduction; F(ab′)₂ is the dimer of two Fab′-fragments, which are connnected by two disulfide-bonds;

(4) Fv, defined as genetechnological modified fragment, containing the variable region of the light chain and the variable region of the heavy chain and being expressed in form of two chains; and

(5) single chain antibody (“SCA”), defined as genetechnological modified molecule, containing the variable region of the light chain and the variable region of the heavy chain, which are genetically fused by an appropriate polypeptide-linker to a single chain molecule.

The procedures for the production of these fragments are insight knowledge in the field (e.g. compare Harlow und Lane, Antibodies: A Laboratory Manual, (1988), Cold Spring Harbor Laboratory, New York).

The term epitope used in this invention means any antigen determinant of antigens, especially such associated with Borreliosis, to which binds the paratope of an antibody. Epitope determinants typically consist of chemically active surface groups of molecules, such as ammino acids or sugar side chains, and usually possess specific characteristics of the three-dimensional structure as well as also specific charge characteristics.

The expert can easily produce monoclonal antibodies in the sense of the invention against the invented proteins and the fragments thereof, or can produce other biological structures, respectively. The common methods under application of hybridom techniques for the production of monoclonal antibodies are wellknown. Immortalised antibody producing cell lines can be produced by means of cell fusion and also by other procedures, such as direct transformation of B-Lymphocytes with onkogenic DNA or transfection with the Epstein-Barr-Virus. See for instance M. Schreier et al., “Hybridoma Techniques”, (1980); Hammerling et al., “Monoclonal Antibodies and T-cell Hybridomas”, (1981); Kennet et al., “Monoclonal Antibodies”, (1980); see also U.S. Pat. Nos. 4,341,761, 4,399,121, 4,427,783, 4,444,887, 4,452,570, 4,466,917, 4,472,500, 4,491,632 and 4,493,890. Groups of monoclonal antibodies or fragments thereof raised against the protein of interest can be screened in the view of different properties, e.g. isotope, epitope, affinity, etc.,. In another form genes can be isolated, coding for monoclonal antibodies of interest, by means of commonly known PCR-procedures known in the field from hybridomas and cloned and expressed in suitable vectors. By use of immunoaffinity procedures monoclonal antibodies are suitable for the isolation and purification of individual proteins, against which they are directed. Independent on whether monoclonal or polyclonal antibodies are dealt with, the invented antibodies have an additional utility since they can be applied as reagents for immunity checks such as RIA, ELISA and similar tests. Furthermore they can be used for isolation of Borreliosis antigens or domains derived from cells or other biological probes. The antibodies might be used, e.g., for the establishment of tests based on tissue culture, in order to find new Borreliosis antigens or new compounds, which can be used to isolate or modify the interaction of Borreliosis antigens and receptors and/or targets.

The humanised or chimeric antibodies can contain parts which derive from two different types (e.g. human constant region and binding region from mice). The parts deriving from different organisms or types may be linked by normal chemical procedures or produced from individual fusion proteins by genetechnological procedures. A DNA, which codes for the proteins of both parts of the chimeric antibody may be expressed as one single fusion protein.

The antibody may specifically bind to one protein or for instance to another biological structure or shows a specifical immunereactivity if in the presence of a heterologous population of proteins and other biological structures performs its biological function by which it can be decided whether this protein or another structure is present. Under the defined binding properties in an immunoassay the defined antibodies bind preferably to a special protein whereas no significant binding occurs to another protein in the sample. Under these conditions a specific binding to a protein demands for an antibody which is selected on the basis of its specifity against a special protein. For the selection of antibodies which show a specific immunoresponse against a special protein different immunotesting procedures may be used. For instance solid-phase-ELISA-Immunotests can be routinely employed for the selection of monoclonal antibodies which show a specific immunoreactivity with an individual protein. Harlow and Lane, Antibodies, A Laboratory Manual, (1988), Cold Spring Harbor Publications, New York, describe Immunotest-procedures and conditions which can be used for the determination of a specific immunoreactivity.

The immunotest concerns a test which uses a test whereby an antibody us employed for the specific binding of an analyte. The immunotest is characterized by specific binding properties which allow to isolate, to specifically test and/or determine quantitatively the analyte by specific binding properties of a special antibody.

According to the presented inovation a vaccination is a manner to avoid Borreliosis associated illnesses. The development of a vaccine implies the identification of factors which cause the infection or are structures or Borreliosis antigens, which are especially detected by the human immune system for the elimination of the cause. Such antigens are often associated with the membrane of the Borreliosis spirochete.

The term protein used in this invention with immunogenic properties of Borreliosis antigens or Borreliosis causing agitans, respectively, implies every protein, polypeptide or peptide, which can serve(1) as antigen for antibodies, which can specifically bind to Borreliosis antigens or Borreliosis-causing agitans, respectively, or (2) by application of a vaccine which can cause a protection against an infection with Borreliosis antigen or Borreliosis causing agitans, respectively. The expert can determine proteins, peptides or polypeptides by usual procedures which show these properties. For instance, these proteins, polypeptides or peptides show 50%, 60%, 70% or 80%, preferably 90%, predominantly 95% and most preferred 98% homology to the proteins, and peptides, respectively which are identified as Borreliosis antigens, whereby for instance, this homology can be determined by the “Smith-Waterman”Homology search-algorithm, e.g. with the program “MPSRCH” (Oxford Molecular) with an affinity gap search by the following parameters: gap open penalty 12, gap extension penalty 1.

The term hybridisation and hybridise in this presented invention is used in context with conventional hybridisation conditions with solutions of 5×SSPE, 1% SDS, 1× Denhardts-solution and hybridisation temperatures of 35° C. to 70° C., preferably at 65° C. After hybridisation washs are done preferably first with 2×SSC, 1% SDS and then with 0,2×SSC at a temperature of 35° C to 70° C., preferably at 65° C. (for definition of SSPE, SSC und Denhardts-solution see Sambrook et al., Molecular Cloning: A Laboratory Manual, 2. edition, Cold Spring Harbor Laboratory Press, Cold Spring Harbor N.Y. (1989)).

Rather preferred are stringent hybridisation conditions, e.g. as cited in Sambrook et al., supra.

The term fragment used in the presented invention implies DNA- and/or protein molecules, which differ from the original sequence by deletion(s), insertion(s), replacement(s) and/or other modifications known in the field as state of the art and imply a fragment of the original amino acid and or nucleotide molecule, whereby it contains the listed properties of the coded sequence of the molecule. Also implied herein are allelic variants and modifications. Procedures which produce the above mentioned alterations of the amino acid and/or nucleotide sequence are known to the experts and described in standard publications of molecular biology, such as in Sambrook et al., supra. The expert is also able to determine whether a protein coded by an altered nucleotide sequence still possesses the above mentioned properties.

The antigens and their fragments especially react specifically against anti-B.garinii-, anti-B. burgdorferi- und/oder anti-B. afzelii antibodies and do not crossreact with other non-Lyme Borreliosis (LM) specific antibodies. They possess a high sensitivity and specifity (their signal frequency is >50%). All proteins (see Table 3) were not known as immuno-relevant Borreliosis antigens.

With the invention presented LM which in Europe is induced by the widely spread strains B. burgdoferi, B. garnii und B. afzelii, can be diagnosed and treated.

Therefore, subject of the invention are agents for diagnosis and/or a diagnostic composition for the proof of Lyme Borreliosis which contain the antigen: glyceraldehyde-3-phosphate-dehydrogenase (GAPDH), oligopeptide permease, oligopeptide ABC transporter periplasmic BP(oppA-2)(Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane associated protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (LDH), P83/100 gene Borrelia burgdorferi, enolase 2-phosphoglycerate Borrelia burgdorferi, flagellin protein Borrelia garinii, hypothetical protein BBE28 Borrelia burgdorferi, DNA directed RNA polymerase (rpoA) homolog, integral outer membran protein p66 (fragment), flagellin (fragment), DNA directed RNA polymerase, integral outer membran protein p66, pyruvate kinase (pyk) homolog, phosphoglycerate kinase (pgk) and/or and/or BBU28760 NID and/or their fragments. In a preferable manner these antigens react specifically with anti-B. garinii-, anti-B. burg-dorferi- and/or anti-B. afzelii-antibodies. The topics of the invention also implies the coding nucleotide sequences of the antigens and/or of the fragments which express the antigens.

The antigens may be isolated and purified from B. burgdorferi, B. garnii, and/or B. afzelii or may represent the coding sequences for the proteins or antigens isolated and purified according to known chemical or genetechnological techniques as recombinant proteins or in form of their genes (DNA, cDNA), including delivered regulation units known to experts, e.g. promoters as well as nucleic acids or nucleic acid sequences, especially in form of plasmids and vectors, and forming proven gene products. The presented invention concerns also a diagnosis procedure for the proof of an acute, chronical or early infection by Borreliosis agitants, where one brings in contact the antibodies directed against Borreliosis and a sample, a fragment thereof or protein with the immunogenic properties, and determines, whether these are bound to the antigen. Thereby the proof is that the antibodies produced by the host can be proven to be directed against the Borreliosis agitant (by means of the invented antigens).

In the diagnosis procedure, e.g. a sample of blood or lymphe is taken, or serum or lymphe are isolated and brought in contact with the invented antigen, and then it is determined whether antibodies from serum are bound to the antigen. The invented diagnosis procedure may be designed as an ELISA, RIA or any other common test procedure, where e.g. the antibody obtained from serum or lymphe can be proven by the aid of a second antibody. Of course in the invented diagnosis procedure the antigen or a fragment thereof may be immobilised, e.g. adsorbed at the wall of a plastic disc so that the specific binding properties are remaining.

The identification of antigens derived from B. garnii, B. burgdorferi and/or B. afzelii is done, e.g. through a combination of high resolution two-dimensional gel electrophoresis with matrix-assisted laser desorption ionization mass spectrometry (MALDI-MS).

The invented antigens are preferably immobilised at a solid carrier in the invented procedure. All known solid phases may function as solid carrier which may be used in form of membranes, gels, test stripes, paper, film, filter, plate or beads.

Through the immobilisation the antigens are brought into a reactive space-limited condition. By immobilisation all methods are implied in the sense of the invention which are understood as limitation of movement of the antigens in a chemical or physical sense. The carrier or the solid phase onto which the antigen is immobilised may also be membranes, nets and/or fibrilles. The immobilisation of the antigen onto the carrier can be directly fixed via a spacer.

Spacer in the sense of the invention may be spacers which form a short molecule chain between the antigen and the carrier. For instance, hydroxilated chains may be used in order to avoid specific hydrophobic interactions. It may also be possible to immobilize the antigens via selected acceptor molecules. At the immobilisation of the antigens with the aid of acceptor molecules the acceptor molecules should provide the necessary properties, e.g. charge, chemically modified groups, and/or immune-, nucleic acid- and/or hybridisation affinities. The immobilisation of the antigens not necessarily must be fixed by a spacer by the aid of acceptor molecules. Of course it may also be provided that the antigen directly can be immobilised to the carrier.

The immobilsation can be done by various methods, e.g. by binding of the antigens with each other, by fixation through a net or a polymer matrix or enclosure by membranes. By the immobilisation the antigens may be reused but can also easily be released of the interaction with the biological sample after the process. They may be applied in much higher local concentrations and in continuous flow-through systems. The binding and the immobilisation of the antigens, respectively, at the carrier may also be achieved by direct carrier bonds or by cross-linking. According to the invention the carrier bond is done either by ionic/adsorptive or by covalent binding. The cross-linking according to the invention is, e.g., a cross-link of the antigens with each other or with other polymers. At the immobilisation by inclusion the antigens are enclosed in gel structures or membranes.

The inclosure of the antigen is also possible, if e.g., the carriers are sintered glass-sponges whereby a concentration of the Borreliosis-antigens is possible. Furthermore the immobilisation at the carrier is possible through insertion into the gel by fixation in Carrageen, by the insertion in polyacrylamide, in alginate, in ENT-polymer on the basis of ceramics by polyamine, and the cross-linking by glutaraldehyde.

Under the condition for a successful covalent fixation of the Borreliose-antigens the presence of functional groups on the carrier may be important. A possible activation procedure, e.g. on dextran gels is the reaction with cyanogen bromide. Depending on the chemical nature of the functional groups, different binding-types can be formed, e.g. ethers, thioethers, esters, etc. Known to the expert are further coupling procedures for the covalent attachment of antigen to agar-, agarose-, and Sephadex-carriers, as well as silanised surfaces of porous glasses. Possible changes of the activity of the antigens can be circumvented or eliminated if the antigen is immobilised to the carrier via a spacer. The spacer provides the necessary flexibility for an optimal binding to the antibody and for the optimal recognition of the binding partner to be analysed.

The invention concerns also the use of the antigens glyceraldehyde-3-phosphate-dehydrogenase (GAPDH), oligopeptide permease, oligopeptide ABC transporter periplasmic BP(oppA-2) (Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane associated protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (LDH), P83/100 gene Borrelia burgdorferi, enolase 2-phosphoglycerate Borrelia burgdorferi, flagellin protein Borrelia garinii, hypothetical protein BBE28 Borrelia burgdorferi, DNA directed RNA polymerase (rpoA) homolog, integral outer membran protein p66 (fragment), flagellin (fragment), DNA directed RNA polymerase, integral outer membran protein p66, pyruvate kinase (pyk) homolog, phosphoglycerate kinase (pgk) and/or and/or BBU28760 NID and/or their fragments and/or the coding nucleic acid sequences of the antigens and/or their fragments for diagnosis and/or the therapy of Lyme Borreliosis.

The invention also concerns the use of the antigens glyceraldehyde-3-phosphate-dehydrogenase (GAPDH), oligopeptide permease, oligopeptide ABC transporter periplasmic BP(oppA-2) (Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane associated protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (LDH), P83/100 gene Borrelia burgdorferi, enolase 2-phosphoglycerate Borrelia burgdorferi, flagellin protein Borrelia garinii, hypothetical protein BBE28 Borrelia burgdorferi, DNA directed RNA polymerase (rpoA) homolog, integral outer membran protein p66 (fragment), flagellin (fragment), DNA directed RNA polymerase, integral outer membran protein p66, pyruvate kinase (pyk) homolog, phosphoglycerate kinase (pgk) and/or BBU28760 NID and/or their fragments and/or the coding nucleic acid sequences of the antigens and/or their fragments for the production of agents for diagnosis and/or therapy of Lyme Borreliosis.

Subject of the invention further is a method for the proof of Lyme Borreliosis, marked by the fact that the antigens glyceraldehyde-3-phosphate-dehydrogenase (GAPDH), oligopeptide permease, oligopeptide ABC transporter periplasmic BP(oppA-2)(Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane associated protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (LDH), P83/100 gene Borrelia burgdorferi, enolase 2-phosphoglycerate Borrelia burgdorferi, flagellin protein Borrelia garinii, hypothetical protein BBE28 Borrelia burgdorferi, DNA directed RNA polymerase (rpoA) homolog, integral outer membran protein p66 (fragment), flagellin (fragment), DNA directed RNA polymerase, integral outer membran protein p66, pyruvate kinase (pyk) homolog, phosphoglycerate kinase (pgk) and/or BBU28760 NID and/or their fragments/or the coding nucleic acid sequences of the antigens and/or their fragments are brought in contact with a biological sample which contains Borreliosis antibodies under conditions which allow for a formation of antigen-antibody complexes and, after an incubation time the complex formation of the antibody with the antigen can be proven.

According to the invention the proof of a Borreliosis-antibody is done preferably in an immune assay, preferably in a solid-phase immunoassay, under direct or indirect coupling of the reaction partner to a well detectable marker substance. Most preferably the proof can be done in an ELISA, a RIA or a fluorescence immunoassay. The procedure of this proofs are welknown to the expert.

For instance, in an ELISA the antigen, in the presented case, e.g. glyceraldehyde-3-phosphate-dehydrogenase (GAPDH), oligopeptide permease, oligopeptide ABC transporter periplasmic BP(oppA-2) (Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane associated protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (LDH), P83/100 gene Borrelia burgdorferi, enolase 2-phosphoglycerate Borrelia burgdorferi, flagellin protein Borrelia garinii, hypothetical protein BBE28 Borrelia burgdorferi, DNA directed RNA polymerase (rpoA) homolog, integral outer membran protein p66 (fragment), flagellin (fragment), DNA directed RNA polymerase, integral outer membran protein p66, pyruvate kinase (pyk) homolog, phosphoglycerate kinase (pgk) and/or BBU28760 NID and/or their fragments/or the coding nucleic acid sequences of the antigens and/or their fragments are directly or indirectly bound to a carrier substance, such as for instance polystyrene. After incubation with the antibodies to be poven, e.g. from the serum of patients, the antigen-bound antibodies are directly or indirectly detected by enzyme-coupled substances. These substances may be antibodies, fragments of antibodies or high affinity ligands such as e.g. avidin which is bound to a biotin-label. As enzymes for instance, peroxidase, alkaline phosphatase, O-galactosidase, urease or glucoseoxidase may be considered. By addition of a chromogenic substrate the bound enzymes and therefore, e.g. the bound antibodies can be determined quantitatively.

Also in a radio-immunoassay the antigen, e.g. glyceraldehyde-3-phosphate-dehydrogenase (GAPDH), oligopeptide permease, oligopeptide ABC transporter periplasmic BP(oppA-2) (Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane associated protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (LDH), P83/100 gene Borrelia burgdorferi, enolase 2-phosphoglycerate Borrelia burgdorferi, flagellin protein Borrelia garinii, hypothetical protein BBE28 Borrelia burgdorferi, DNA directed RNA polymerase (rpoA) homolog, integral outer membran protein p66 (fragment), flagellin (fragment), DNA directed RNA polymerase, integral outer membran protein p66, pyruvate kinase (pyk) homolog, phosphoglycerate kinase (pgk) and/or BBU28760 NID and/or their fragments and/or the coding nucleic acid sequences of the antigens and/or their fragments are directly or indirectly bound to a carrier substance, e.g. polystyrene. After incubation with the antibody to be proven, e.g. from serum of patients, the antigen-bound antibodies will be determined by substances which carry a radiolabel, e.g. ¹²⁵I. These substances may be antibodies, fragments of antibodies or high affinity ligands, such a avidin, which binds to a biotin-label. The bound radioactivity may be qunatitatively determined by a suitable detector instrument.

According to the same procedure the antigen-bound antibodies can be measured by fluorescent immunoassays with substances, e.g. by fluorescence-labeling carrying, e.g. fluorescein-isothiocyanate (FITC). These substances may be antibodies, fragments of antibodies or high affinity ligands, such as avidin, binding to a biotin label. The amount bound of the fluorescent dye then will be qunatitatively determined by an appropriate fluorescence spectrophotometer.

Of course it is possible also to proof the Lyme Borreliosis by biochips, e.g. by:

a) Immobilisation of specific antibodies against the baterial protein-antigen which may also be done on a chip in order to prove the bacterium directly in humans;

b) Immobilisation of specific binding peptides and/or nucleic acids, which can be obtained by the evolutionary method, especially by the “phage-display” method and directly prove the bacteria;

c) Immobilisation of characterised proteins or the peptide fragments thereof on the chip in order to prove the antibodies of the patients preferably by the proof of Borreliosis-antibodies in serum.

Biological sample in the context of the invention are fluids of animals or humans like e.g. blood, plasma, urine, liquor, and synovialfluid. The proof of Borreliosis is preferably executed with humans by the proof of Borreliosis-antibodies in serum.

Subject of the invention further is the Borreliosis vaccine or a pharmaceutical composition, respectively which contains e.g., glyceraldehyde-3-phosphate-dehydrogenase (GAPDH), oligopeptide permease, oligopeptide ABC transporter periplasmic BP(oppA-2)(Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane associated protein p66 percursor Borrelia burgdoferii, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (LDH), P83/100 gene Borrelia burgdorferi, enolase 2-phosphoglycerate Borrelia burgdorferi, flagellin protein Borrelia garinii, hypothetical protein BBE28 Borrelia burgdorferi, DNA directed RNA polymerase (rpoA) homolog, integral outer membran protein p66 (fragment), flagellin (fragment), DNA directed RNA polymerase, integral outer membran protein p66, pyruvate kinase (pyk) homolog, phosphoglycerate kinase (pgk) and/or BBU28760 NID and/or their fragments/or the coding nucleic acid sequences of the antigens and/or their fragments which contain the coding DNA of these proteins or the fragments thereof.

The vaccine according to the invention may contain defined bacterial products, immortilised vaccine, vital vaccine, synthetic peptides, recombinant proteins, deletion mutants, recombinant vaccinated strains, and/or anti-idiotypic vaccines.

The vaccination with defined bacterial products would be a preferable agent for an easy vaccination against the Borreliose-agitants, or Lyme Borreliosis, respectively. The immune response may contain an immune response against the agitants or against the agitant's products. The vaccination with immortalised vaccine in the sense of the invention comprises the vaccination with dead Borreliosis bacteria, whereby the agitant may be the complete organism as well as individual antigenic structures thereof. The vaccination with vital vaccine preferably leads to a strong induction of sufficient resistence against Lyme Borreliosis. Advantageously it is possible, also to vaccinate with synthetic peptides, which may have the advantage to comprise the smallest possible unit of the bacterium.

An antigen or a molecule, respectively which carries the epitope responsible for the protective immunoresponse may advantageously contain further additional regions. At the synthesis of the peptide responsible for the protection one can use this isolate from the injurious sequence areas. With advantage it is possible to couple the peptide to a carrier molecule. Of course it is also possible to synthetise artificial polypeptides which consist of repeated subunits of protective peptides. The vaccination with recombinant antigens, and peptides or proteins, respectively, in the sense of the invention allows advantageously to produce the suitable peptides in big amounts and thereby accertains the production of sufficient amounts of vaccines even with severe or not at all cultivatable bacterial strains or agitant modifications, respectively. With advantage, in this manner any contaminations by injurious products can be excluded if they are not coded for by the same gene. By appropriate means the genome of the bacterium can be changed in a manner that cannot lead to an illness in the host. This can be reached preferably by gene deletions, which are necessary for the virulence or survival in the host. Since this metabolites are essential for the bacterium, but not provided by the organism of the patient the agitant dies in the patient after the reserve products are consumed. The survival time in the host however is long enough to induce a protective immune response. Recombinant vaccines are considered with success if the protective antigen or the Borreliosis agitant is available in recombinant form, but not able alone to induce the protection. This is of importance where the protection is carried out mainly by various lymphe populations. In this case corresponding the gene responsible for the protective antigen may be cloned into a suitable vector, which allows for a suitable immune response against the recombinant molecule. The concept of an anti-idiotypic vaccine in the sense of this invention is based on anti-idiotypic antibodies which are recognised by protective antibodies which are able to stimulate their synthesis. For instance, in the case of carbohydrate antigens the production of molecular antibodies may preferably be less difficult as the production of the antigen itself. Further the immunoresponse caused by anti-idiotypic antibody may be controlled by another method than that of the normal antigen. Therefore, the anti-idiotypic vaccine or their vaccines, respectively may also provide a sufficient protection in small children. Of course it is possible, also to vaccinate with antibodies directed against anti-antigen-antibodies.

The presented invention also concerns an immunogenic composition, preferably a vaccine, which contains the above listed Borreliose-antigens, a fragment thereof and/or protein with the immunogenic properties, or alternatively antibodies which are directed against the antigens. The vaccine invented contains additionally a pharmaceutically neutral carrier. Suitable carriers and the formulation of such a vaccine are known to the expert. Suitable carriers are, e.g. phophate-buffered saline solutions, water, emulsions, e.g. oil/water-emulsion, detergents, sterile solutions, etc. The application of the vaccine may be by oral, parenteral, or subcutan application, e.g. intradermal, subcutan or intramuscular. The suitable dosage will be determined by the doctor and depends on various factors, e.g. depends on the manner of application, from the age and weight of the recipient, etc. Typically the dosage is in the range of 1 μg bis 200 μg per patient. In children the dosis is appropriately lower to 5 μg, and in hemodialysis patients is is appropriately higher to about 30-300 μg.

The description is examplified in the following by an example without restricting the invention to only this.

### EXAMPLE

Identification of Borreliose Antigens

Methods

Cells of five different strains of Borreliosis bacteria (B. burgdorferi B31, B. garinii 20047, B. garinii BITS, B. garinii VS286 and B. afzelii PKO (VS461), isolated and multiplied from skin-punches and ticks, were lysed under frozen conditions and centrifuged in an ultracentrifuge at 4° C. The supernatants containing the entire protein mixtures were applied and separated on high resolution two-dimensional polyacrylamide gels (2DE-Gels) (according to Jungblut P., Grabher, G., Stöffler, G., Electrophoresis 20, 3611-3622, 1999; and Klose und Kobaltz, 1995, Electrophoresis 16, 1043-1049).

Analytical and preparative 2DE-gigant gels (24×32 cm) were stained by silver staining solution or by Coomassie-Blue solution and scanned for documentation (see FIGS. 1, 2, 3 , 4 and 5 of the different Borreliose strains).

In an analogous manner 2DE-gels of all cell lysates were transferred without staining onto PVDF-membranes and incubated with sera each of nine different IgG positive Neuroborreliosis patients (Sera 2-10), all late manifestations (according to Jungblut, P., Grabher, G., Stöffler, G., Electrophoresis 20, 3611-3622, 1999). Those proteins which reacted positively against these sera on the immuno-stained gels were named by numbers in FIGS. 1-5 and were labeled by + (plus) in Table 1; those which were negative were given a − (minus).

The proteins were separated in a preparative 2DE-gel, stained by Coomassie Blue, exised as spots and digested with trypsin. The generated tryptic peptides were eluted, desalted and analysed by MALDI-mass spectrometry. By comparison of the mass data (“mass fingerprints”) with the exsisting protein and gene databases (SWISSPROT, PIR) the proteins were identified by the use of the mass comparison program Mascot and marked by numbers in the figures (see also Table 1, 2, and 3).

In Table 2 those proteins which reacted positively and with which of the sera are listed. Serum 5 was very weak and therefore reacted only with protein 1, 3, 6, 19 derived from B. burgdorferi, and gave a positive reaction with protein 1, 19 from B. garinii VS286, and with protein 24 from B. garinii 20047. It is likely that also the other proteins with negative reaction would yield a positive reaction with other sera of patients if more sera are measured.

In Table 3 the names of the identified proteins, and their corresponding gene names are listed together with their accession numbers in the corresponding databases (S.Pt: SwissProt-Database; PIR BLS: PIR Database). Furthermore, in Table 3 the possible cell- or plasmide-positions of the genes are given, and were it is known also the functions of these proteins are given (Fraser et al., Nature 390, 580-586, 1997).

For each of the proteins the scores for significant identification (score) are given (threll value larger/equal 63), and the peptide sequence coverage in % coverage), the number of amino acid residues contained in the protein (no. a.a.), their molecular mass (Mw) and the reference (ref).

Results:

1. All the proteins positively detected in the immunoblots were identified by mass spectrometry as existing in the list of the gene sequences in the database of the organisms Borrelia burgdorferi, B. afzelii and/or B. garinii (see Table 1-3).

2. Not all the identified proteins were listed in the published gene database (according to Fraser et al., Nature, 390, 580-586, 1997) of the organism B. burgdorferi. These four proteins are marked by * in Table 3.

3. These authors indicate in their publication that during genome analysis they found some unclear regions unresolved so far. This is the cause for and explains the low scores (below 63) for these proteins (see Table 3).

4. Those proteins which reacted positively with all nine sera of patients were identified in B. burgdorferi, and in part also in all other three investigated B. garinii and in the B. afzelii-strain (see Table 2). Some of the sera were rather weak; therefore these sera showed only a positive reaction for the strongest protein spots in the gel.

The following proteins were identified:

| | |
|---|---|
| Spot 1 | Oligopeptide ABC transporter periplasmic |
| | BP(oppA-2) (Bb) |
| Spot 1a | Glycosyl transferase IgtD homolog |
| Spot 1b | Heat shock protein 90 |
| Spot 1c | VLSE fragment |
| Spot 2 | (U76406) putative v1s rec. casette V1s6 |
| | Borrelia burgdorferi |
| Spot 3 | Flagellin protein Borrelia garinii |
| Spot 4 | (AE001578) conserved hypothetical |
| | protein Borrelia burgdorferi |
| Spot 5 | Membrane associated protein p66 |
| | percursor Borrelia burgdoferii |
| Spot 6 | Oligopeptide ABC transporter periplasmic |
| | BP (oppA-4) (Bc) |
| Spot 7 | Fructose-biphosphate aldose (fba) |
| | Borrelia burgdorferi |
| Spot 8 | DNAK Heat Shock Protein 70 Borrelia |
| | burgdorferi |
| Spot 9 | orfE Borrelia burgdorferi |
| Spot 10 | Outer surface protein B precursor |
| | Borrelia burgdorferi |
| Spot 10a | L-lactate dehydrogenase (ldh) |
| Spot 12 | P83/100 gene Borrelia burgdorferi |
| Spot 13 | Enolase 2-phosphoglycerate Borrelia |
| | burgdorferi |
| Spot 15 | Flagellin Protein Borrelia garinii |
| Spot 16 | Hypothetical protein BBE28 Borrelia |
| | burgdorferi |
| Spot 18 | DNA directed RNA polymerase (rpoA) |
| | homolog |
| Spot 20 | P66 protein (fragment) |
| Spot 27 | Flagellin (fragment) |
| Spot 28 | DNA directed RNA polymerase |
| Spot 31 | Integral outer membran protein p66 |
| Spot 35 | Pyruvate kinase (pyk) homolog |
| Spot 36 | Phosphoglycerate kinase (pgk) |
| Spot 38 | BBU28760 NID. |

Among these proteins are four hypothetical proteins which so far have only been sequenced as open reading frames (ORFs) but have not been identified and characterized as proteins on the protein level, and two were not identified at all (protein spot 2, spot 4, spot 9, spot 16 and spot 38).

5. The vaccine developed in America (The New England Journal of Medicine (1998), Vol. 339, No. 4) is only directed against the immunogenic protein OspA (“outer surface lipoprotein”), therefore the identification of the antigens specified in Table 2 and 3 are valuable for the diagnosis and vaccine development of the Borreliosis diseases.

6. All positively identified proteins and their fragments listed in Table 2 and 3 can be applied to the design ofprotein or antibody chips for a fast screening of Borreliosis infections.

7. The positively identified antigens confirm those proteins characterized by another method (according to Jungblut et al., 1999), namely proteins p83/100, flagellin, oligopeptide permease opp-A.

In the publication of Jungblut et al.(1999) these proteins were identified in the following manner: They were isolated from small 2DE-gels, exiced from the gel and analysed by MALDI-mass spectrometry and by special antibodies. The diagnosis by patient sera were made by ELISA-tests as done under routine laboratory conditions, or in one-dimensional gels by staining with rabit or human antisera, respectively. Under these conditions two new antigens, namely GAPDH und ABC transporter oligopeptide permease, were identified besides the akready known OspA.

Legends

FIGS. 1-5:

High Resolution Two-Dimensional Polyacrylamide Gel (According to Klose und Kobalz, 1975) of an Entire Protein Mixture of the Lysate of Various Borrelia Cells

1. Dimension IEF-Gel (separation according to charge of the proteins) and 2. dimension SDS-Gel (separation according to size of the proteins); size of the gels 24×30 cm; silver staining. All protein spots marked in FIGS. 1-5 were identified by mass spectrometry of the generated peptides. The scales list the pH-values and masses of the proteins.

Table 1: Important Borrelia proteins from the 2DE-gels Analytical 2DE-Gele derived from 5 different Borrelia strains were produced and stained by silver solution.

+ (marked by plus), protein spots contained in the gels of the respective strains which reacted positively with the sera employed.

− (marked by minus), protein spots contained in the gels of the respective strains which could not be identified with the sera employed (either non existing, expressed in too low amounts, or present at other parts of the gel, respectively).

Tabelle 2: Immunological Test Against Nine Sera of Patients

Analytical gels were produced from the different Borrelia strains and transferred to PVDF-membranes without any staining. All these membranes were treated and incubated with nine different sera from Neuroborreliosis-patients

Protein spots which yielded a positive immunological reaction are listed in the table.

From the results it follows that the following protein spots were identified in all strains of B. burgdorferi und B. garinii tested (see Table 1) and these spots also immuno-reacted positively with the sera employed:

| | |
|---|---|
| Spot 1 | Oligopeptide ABC transporter periplasmic |
| | BP (oppA-2) (Bb) |
| Spot 1a | Glycosyl transferase IgtD homolog |
| Spot 1b | Heat shock protein 90 |
| Spot 1c | VLSE fragment |
| Spot 2 | (U76406) putative v1s rec. casette V1s6 |
| | Borrelia burgdorferi |
| Spot 3 | Flagellin protein Borrelia garinii |
| Spot 4 | (AE001578) conserved hypothetical |
| | protein Borrelia burgdorferi |
| Spot 5 | Membrane assoc. protein p66 percursor |
| | Borrelia burgdoferi |
| Spot 6 | Oligopeptide ABC transporter periplasmic |
| | BP (oppA-4) (Bc) |
| Spot 7 | Fructose-biphosphate aldose (fba) |
| | Borrelia burgdorferi |
| Spot 8 | DNAK protein heat shock protein 70 |
| | Borrelia burgdorferi |
| Spot 9 | orfE Borrelia burgdorferi |
| Spot 10 | Outer surface protein B precursor |
| | Borrelia burgdorferi |
| Spot 10a | L-lactate dehydrogenase (Idh) |
| Spot 12 | P83/100 gene Borrelia burgdorferi |
| Spot 13 | Enolase 2-phosphoglycerate Borrelia |
| | burgdorferi |
| Spot 15 | Flagellin protein Borrelia garinii |
| Spot 16 | Hypothetical protein BBE28 Borrelia |
| | burgdorferi |
| Spot 18 | DNA direct. RNA polymerase (rpoA) |
| | homolog |
| Spot 20 | P66 protein (fragment) |
| Spot 27 | Flagellin (fragment) |
| Spot 28 | DNA direct. RNA polymerase |
| Spot 31 | Integral outer membran protein p66 |
| Spot 35 | Pyruvate kinase (pyk) homolog |
| Spot 36 | Phosphoglycerate kinase (pgk) |
| Spot 38 | BBU28760 NID. |

Table 3:

Identification of the Immune-Positive Protein Spots by Mass Spectrometry:

Abbreviations:

| | |
|---|---|
| Spot X: | Number of proteins in silver- |
| | stained 2DE-gel. |
| Name: | Name of the identified protein (see |
| | TABLE 2) |
| Ac.S.P. | Accession number in the SwissProt- |
| | Database |
| Ac.PIR.BLS. | Accession number in the PIR BLAST |
| | Database |
| Gene Ident. | Corresponding gene name of the |
| | organism Borrelia burgdorferi |
| Position/Function | Position on one of the plasmides of |
| | B.burgdorferi; function of the |
| | protein in metabolic cycle |
| Score | Score value for significance |
| | (probability value by the program |
| | Mascot (=larger/equal 63) |
| % Cover. | Sequence coverage of the peptide |
| | masses in comparison with protein |
| | sequence in the database |
| No a.a | number of amino acid residues in |
| | the protein |
| Mw | Molecular mass of the proteins |
| Ref. | references |
| * | Hypothetical proteins, whose genes |
| | are contained in the NCBI database, |
| | but which were not yet identified |
| | as proteins |

TABLE 1
Spot table for 5 Different species of Borrelia

| | Borrelia burgdorferi B31 | Borrelia garinii BITS | Borrelia garinii VS286 | Borrelia garinii 20047 | Borrelia afzelii Pko |
|---|---|---|---|---|---|
| Spot 1 | | | | | — |
| Spot 2 | | | | | — |
| Spot 3 | | — | | | |
| Spot 4 | | | | | — |
| Spot 5 | | — | | — | |
| Spot 6 | | — | — | — | — |
| Spot 7 | | — | — | | — |
| Spot 8 | | | | | |
| Spot 9 | | | | | — |
| Spot 10 | | — | | — | — |
| Spot 11 | | — | | | |
| Spot 12 | | | | | |
| Spot 13 | | | | | — |
| Spot 14 | | | | | — |
| Spot 15 | | | | | |
| Spot 16 | | | | | — |
| Spot 17 | | — | — | | — |
| Spot 18 | | — | | | |
| Spot 19 | | | | | — |
| Spot 20 | | | — | | — |
| Spot 21 | | — | | | |
| Spot 22 | | — | — | | |
| Spot 23 | | | | | |
| Spot 24 | | | | | |
| Spot 25 | | | | | — |
| Spot 26 | — | — | — | — | |
| Spot 27 | | | | | |
| Spot 28 | — | — | — | — | |
| Spot 29 | | — | | — | |
| Spot 30 | — | — | — | — | |
| Spot 31 | | | | | |
| Spot 32 | | — | — | | |
| Spot 33 | | | | | — |
| Spot 34 | | | | | |
| Spot 35 | | | | | |
| Spot 36 | | | — | | — |
| Spot 37 | | | — | | — |
| Spot 38 | | | | | |
| Spot 39 | | | | | |

TABLE 2
Antigenic spots

| | | | | |
|---|---|---|---|---|
| Sera 2,3,4,5,6,7,8,9,10 | Sera 1,2,3,4,6,7,8,9,10,11 | Sera 1,2,4,5,6,8,9,10 | Sera 2,3,4,6,7,8,10 | |
| Sera 2,3,4,6,7,8,9,10 | Sera 2,8,10 | Sera 2,6,9,10 | Sera 6,8,10 | |
| Sera 2,3,4,5,6,7,8,9,10 | Sera 18,9,10 | Sera 1,6,7,8,10 | Sera 2,3,6,7,8,10 | Sera 1,2,6,7 |
| Sera 3,4,5,6,7,8,10 | Sera 2,6,8,10 | Sera 2,6,7,8,10 | Sera 2,7,8,10 | |
| Sera 2,7,9 | | Sera 8 | | Sera 1,2,7,10 |
| Sera 5,6 | | | | |
| Sera 6,7, | Sera 2,6,8,9,10 | | | Sera 1 |
| Sera 6,7,8 | Sera 2,7,10 | Sera 1,2,6,7 | Sera 1,7,8 | Sera 1,10 |
| Sera 6 | Sera 1,2,6,8,9,10 | Sera 1,2,8,9 | Sera 1,2,8 | |
| Sera 6 | | | | |
| Sera 2,6 | | Sera 8 | Sera 8 | Sera 2 |
| Sera 6,8 | Sera 1,2,6,7,8,9,10 | Sera 1,2,6,8,10 | Sera 1,2,6,7,8,10 | Sera 1,7,8 |
| Sera 6,8 | Sera 6,9 | | | Sera 1,2,6, |
| Sera 6,7,8 | | | Sera 8 | |
| Sera 2,6,8,9,10 | Sera 1,3,6,7,8,10 | Sera 1,2,6,7,8,10 | Sera 2,7,8,10 | Sera 1,2,4,6 |
| Sera 6,8 | Sera 2,6 | Sera 8 | Sera 2,7,8,10 | |
| Sera 6,8 | | | Sera 8 | |
| Sera 3,6,8,9, | Sera 2 | | Sera 2 | Sera 7 |
| Sera 2,4,5,6,7,8,9,10 | Sera 1,2,3,4,8,10 | Sera 1,2,4,5,8,9,10 | Sera 2,3,4,6,8,10 | Sera 10 |
| Sera 6 | Sera 1,2 | | | |
| Sera 2 | Sera 1,3 | Sera 2,8 | | Sera 6,10 |
| Sera 3,6 | | | Sera 8 | Sera 2,7,8 |
| Sera 2 | Sera 1,3,10 | Sera 2,8 | Sera 8 | |
| Sera 2 | Sera 1,3,8,10 | Sera 2,8 | Sera 5,8 | |
| Sera 6,8,9,10 | Sera 1,2,6,8,9 | Sera 8,9,10 | Sera 6,7,8,10 | |
| | | | | Sera 1,2,7 |
| Sera 8,10 | | Sera 2,8,10 | Sera 8 | Sera 1,2 |
| | | | | Sera 2,3,6 |
| | | | | Sera 2,4 |
| | | | | Sera 1,2,4 |
| Sera 7 | | | Sera 8 | Sera 7 |
| Sera 7 | Sera 2,9 | Sera 8 | Sera 2,8 | |
| | Sera 6,7,8,9 | Sera 1,2,8 | | |
| | Sera 1,9 | Sera 8 | Sera 8 | |
| | Sera 2,6 | | Sera 1 | |
| | Sera 2,6, | | | |
| Sera 2,3,4,5,6,7,8,9,10 | Sera 1,2,6,8,9,19 | Sera 2,6,9,10 | Sera 2,7 | |
| | Sera 8 | | Sera 8 | |

TABLE 3
Identified proteins

| | | | | | | | | | |
|---|---|---|---|---|---|---|---|---|---|
| Olig.pept. ABC transporter | | H70140 | BB329 | Transp. & Bind. prot. | 165 | 43% | 528 | 60625 | a |
| periplasmic BP(oppA-2) (Bb). | | | | aa.pep.amines. | | | | | |
| Glycosyl transferase lgtD homolog | | C70171 | | | 32 | 10% | 358 | 42079 | a |
| Heat shock prot. 90 | | gi:7441887 | BB560 | Cell Div. Chaperons | 74 | 6% | 650 | 75377 | a |
| VLSE fragment | | Q9RF38 | | | 45 | 32% | 221 | 21235 | s |
| (U76406) putative v1s rec. | | gi 2039285 | | put.v1srec.cass. | 57 | 20% | 190 | 18635 | |
| casette V1s6 Borr. burgdfr. | | | | v1s6 Borr. burgdf. | | | | | |
| Flagellin Protein Bor. garn. | | gi 939802 | (BAA09679) | | 138 | 7% | 329 | 34870 | h,i,j,l |
| (AE001578) conserved | | gi 6382240 | BBM10 | cp32-6 | 36 | 19% | 102 | 21596 | |
| hypothetical prot. Bor. burgdf. | | | | | | | | | |
| Membrane assoc. Prot. | | B70175 | BB603 | Cell envip./membrs. | 111 | 21% | 618 | 68130 | a,b |
| p66 percursor Borrellia burgdf. | | | | lipoprots. & porins | | | | | |
| Olig.pept. ABC transporter | | gi 7442333 | BBA34 | Transp. & Bind. prot. | 205 | 6% | 530 | 60728 | g,m |
| periplasmic BP (oppA-4)(Bc). | | | | lp54 | | | | | |
| Fructose-bisphosphate aldose | O51401 | D70155 | BB445 | Energ. Metabol. | 161 | 39% | 359 | 39955 | a,c |
| (fba) Borr. burgdfr, | | | | Glycolysis | | | | | |
| DNAK Prot. HeatShockProt. 70 | | gi 118722 | BB264;518 | Cell Div. Chaperons | 238 | 6% | 653 | 69233 | k,n |
| Borr. burgdfr. | | | | | | | | | |
| orfE Borrelia burgdrf. | | gi 2935198 | | cp32-4 | 51 | 90% | 11 | 1237 | |
| Outer surface prot B precursor | P17739 | S06915 | BBA16 | Cell envip./lp54 | 214 | 52% | 296 | 31756 | a,d,e |
| Borr. burgdf. | | | | | | | | | |
| L-lactate dehydrogenase (ldh) | | G70110 | BB087 | Energ. Metabolism | 37 | | | 34582 | |
| P83/100 gene Borr. burgdf. | Q45012 | Q45012 | BB744 | Cell envip./srfc. | 80 | 13% | 679 | 77455 | f |
| | | | | plysac. & antigens | | | | | |
| Enolase 2phosphoglycerat | O51312 | ENO BORBU | BB337 | Energ. Metabolism | 71 | 21% | 433 | 47260 | a,g |
| Borr. burgdf. | | | | Glycolysis | | | | | |
| Flagellin Protein Bor. garn. | P70890 | gi:4432995 | (BAA09637) | | 64 | 20% | 235 | 30692 | h,i,o |
| Hypothetical prot BBE28. | | gi:7463507 | PlasmidE | lp25 | 40 | 24% | 50 | 5980 | a |
| Borr. burgdf. | | | | | | | | | |
| DNA direct. RNA polymerase | | E70162 | | | 64 | 16% | 345 | 38661 | a |
| (rpoA) homolog | | | | | | | | | |
| P66 protein (fragment) | Q44944 | Q44944 | | | 67 | 45% | 80 | 8757 | b |
| Flagellin (fragment) | | O31321 | | | 41 | 17% | 151 | 15689 | r |
| DNA direct. RNA polymerase | | C70148 | | | 27 | | | | |
| Integral outer membr. protp 66 | | O51954 | | Membrane prot. | 76 | 9% | 598 | 66030 | P |
| Pyruvate kinase(pyk)homolog | | C70143 | BB348 | Energ. Metabolism | 106 | 19% | 477 | 52999 | a |
| | | | | Glycolysis | | | | | |
| Phosphoglycerat kinase (pgk) | | H70106 | BB056 | Energ. Metabolism | 82 | 24% | 393 | 42319 | a |
| | | | | Glycolysis | | | | | |
| BBU28760 NID | | AAB53930 | BB057 | Energ. Metabolism | 82 | 23% | 335 | 36268 | q |
| | | | | Glycolysis | | | | | |

a) Fraser C M, Casjens S, Huang W M, Sutton G G, Clayton R, Lathigra R, White O, Ketchum K A, Dodson R, Hickey E K, Gwinn M, Dougherty B, Tomb J F, Fleischmann R D, Richardson D, Peterson J, Kerlavage A R, Quackenbusch J, Salzberg S, Hanson M, van Vugt R, Palmer N, Adams M D, Gocayne J, Venter J C, et al.. “Genomic sequence of a lyme disease spirochaete, Borrelia burgdorferi.“ Natare 1997 Dec. 11: 390
b) Bunixis J., Noppa L., Bergstroem S. “Molecular anaylsis of a 66 kDa protein (p66), associated with the outer membrane of lyme disease Borrelia species” FEMS Microbiol. Letl. 131:139-145 (1995)
c) Coleman, J. L.: Benach, J. L “Identification and characterization of an endoflagellar antigen of Borrelia burgdorferi” J. Clin. Invest. (1989) 84:322-330.
d) Bergstroem S., Bundoc V., Barbour A. G. “Molecular analysis of a linear plasmid-encoded major surface proleins, Osp A and Osp B, of the Lyme disease spirochaete Borrelia burgdorferi,” Mol. Microbiol. 3:479-486 (1989).
e) Caporale D. A., Kocher T. D. “Sequence variation in the outer-surface protein genes of Borrelia burgdorferi.” Mol. Biol. Evol. II:51-64 (1994).
f) Roessler D., Eiffert H., Jauris-Heipke S., Lelmeri G., Preac-Mursic V., Teepe J., Schlon T., Soutscbek. E., Wilske B. “Molecular and immunological characterization of the p38/100 protein of various Borrelia burgdorferi sensu lato strains.”, Med. Microbiol. Immonol. 184: 23-32 (1995).
g) Bono J. L., Tilly K., Stevenson B., Hogan D., Rosa P. “Oligopeptide permease in Borrelia burgdorferi: putative peptide- binding components encoded by both chromosomal and plasmid loci.” Microbiology 144: 1033-1044 (1998).
h) “Direct Submission” DDBJ/EMBL/GENEBANK 13.7.1995
i) Fukimaka, M., Okada K., Nakao M., Korishi, T. “Phylogenetic analysis of borrelia species based sequences and its application for molecular typic borrelise” Int. J. Syst. Bacteriol. 46 (2), 416-421 (1996).
j) Miyamota, K., Sato, Y., Okada, K., Fukimaga, M. “Competence of a migratory bird, red-bellied thruchrysolaus as an reservoir for the lyme disease in Japan” Int. J. Syst. Bacteriol. 46(4), 898-905 (1996).
k) Anzola, J., Luft, B. J., Gorgone, G., Dattwyler, R. I., Labesmaa, R. and Peltz, G. “Borrelia Burgdorferi HSP70 Homolog: Characterization of immunoreactive stress protein”. Infect. Immun. 60 (9), 3704-3713 (1992).
l) “ Zhang, J. -R., Hardham J. M., Barbour A. G. and Norris S. Antigenic variation in lyme disease borreliae by pro recombination of VMP-like sequsnce casettes.“ Cell 89 (2), 275-285 (1997).
m) Direct submission 22.04.1997,Bono, J. L.
n) Tilly, K., Hauser, R., Campell, J. and Ostheimer, G. “Isolation of dnaJ, Dank, and grpE homologues from Borrelia burgdorferi and complementations of Escherichia coli.“ Mol. Microbiol. 7 (3), 359-369 (1993)
o) Fukunako, M. and Koreki, Y. Int. “A phylogenetic analysis of Borrelia burgdorferi sensu lato isolates associated with lyme disease in Japan by flagellin gene sequence determination.“ J. Syst. Bacteriol. 46(2), 416-421 (1996).
p) Bunikis J., Luke C. J., Bunikiene E., Bergstrom S., Barbour A. G., J. Bacteriol. 180:1618-1623 (1998).
q) Anda, P., Gebbia, J. A., backenson, P. B., Coleman, J. L. and Benach, J. L. “A glyceraldehyde-3-phosphate dehydrogenase homolog in Borrelia burgdorferi and Borrelia.” Infect Immun. 64 (1), 262-268 (1996).
r) Curtin S. M., Maggs A. D F. Carter P. E., Pennington T. H. Submitted October 1997 to the EMBL/Genebank/DDBJ databases.
s) Iyer R., Hardham J. M., Wormser G. P., Schwartz I., Norris S. J. Submitted November 1999 to the EMBL/Genebank/DDBJ databases.

1. Agents for diagnosis of Lyme Borreliosis comprising the antigens glyceraldehyde-3-phosphat-dehydrogenase (GAPDH), oligopeptide-permease, oligopeptide ABC transporter periplasmic BP(oppA-2)(Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferi, membrane assoc. protein p66 percursor Borrelia burgdoferi, oligopeptide ABC transporter periplasmic BP (oppA-4)(Bc), fructose-biphosphate aldose (fba) Borrelia burgdorferii, DNAK protein heat shock protein 70 Borrelia burgdorferi, orfE Borrelia burgdorferi, outer surface protein B precursor Borrelia burgdorferi, L-lactate dehydrogenase (ldh), P83/100 gene Borrelia burgdorferi, enolase 2-phosphoglycerate Borrelia burgdorferi, flagellin protein Borrelia garinii, hypothetical protein BBE28 Borrelia burgdorferii, DNA direct. RNA polymerase (rpoA) homolog, P66 protein (fragment), flagellin (fragment), DNA direct. RNA polymerase, integral outer membrane protein p66, pyruvate kinase (pyk) homolog, phosphoglycerat kinase (pgk) and/or BBU28760 NID and/or their fragments and/or for the antigens and/or fragments nucleotide coding sequences.

2. Agents according to claim 1, characterised in that the specific reaction of the antigens with the anti-B. garnerii, anti-B. burgdorferi- and/or anti-B. afzelii-antibodies.

3. Agents according to claim 1 or 2, characterised in that the antigens are immobilized to a solid carrier.

4. Use of the antigens dehydrogenase (GAPDH), oligopeptide-permease, oligopeptide ABC transporter periplasmic BP(oppA-2)(Bb), glycosyl transferase IgtD homolog, heat shock protein 90, VLSE fragment, (U76406) putative v1s rec. casette V1s6 Borrelia burgdorferi, flagellin protein Borrelia garinii, (AE001578) conserved hypothetical protein cp32-6 Borrelia burgdorferii, membrane assoc. protein p66 percursor Borrelia burgdoferi, oligopeptide ABC transporter periplasmic BP (oppA-4)(Bc), fructose-biphosphate aldose (fba) Borrelia burgdorferii, DNAK protein heat shock protein 70 Borrelia burgdorferii, orfE Borrelia burgdorferi, outer surface protein B precursor Borrelia burgdorferi, L-lactate dehydrogenase (ldh), P83/100 gene Borrelia burgdorferi, enolase 2-phosphoglycerate Borrelia burgdorferi, flagellin protein Borrelia garinii, hypothetical protein BBE28 Borrelia burgdorferi, DNA direct. RNA polymerase (rpoA) homolog, P66 protein (fragment), flagellin (fragment), DNA direct. RNA polymerase, integral outer membrane protein p66, pyruvate kinase (pyk) homolog, phosphoglycerate kinase (pgk) and/or BBU28760 NID and/or their fragments and/or for the antigens and/or fragments nucleotide coding sequences for the production of an agent for the diagnosis and/or therapy of Lyme Borreliosis.

5. Method for the proof of Lyme Borreliosis, characterised in that an agent according to one of claims 1 to 3 of a biological sample, which contains the Borreliosis antibodies, under conditions which allow the contact and the formation of antigen-antibody-complexes, and the proof of complex formation.

6. Method according to claim 5, characterised in that the biological sample is a body fluid.

7. Method according to claim 5 or 6, characterised in that the proof is performed in an ELISA assay.

8. Borreliosis vaccine characterised by comprising glyceraldehyde-3-phosphate-dehydrogenase (GAPDH), oligopeptide-permease, oligopeptide ABC transporter periplasmic BP(oppA-2)(Bb), glycosyl transferase IgtD homolog, heat shock protein 90, VLSE fragment, (U76406) putative v1s rec. casette V1s6 Borrelia burgdorferi, flagellin protein Borrelia garinii, (AE001578), conserved hypothetical protein cp32-6 Borrelia burgdorferii, membrane assoc. protein p66 percursor Borrelia burgdoferi, oligopeptide ABC transporter periplasmic BP (oppA-4)(Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (ldh), P83/100 gene Borrelia burgdorferii, enolase 2-phosphoglycerate Borrelia burgdorferi, flagellin protein Borrelia garinii, hypothetical protein BBE28 Borrelia burgdorferi, DNA direct. RNA polymerase (rpoA) homolog, P66 protein (fragment), flagellin (fragment), DNA direct. RNA polymerase, integral outer membrane protein p66, pyruvate kinase (pyk) homolog, phosphoglycerate kinase (pgk) and/or BBU28760 NID and/or their fragments and/or antibodies and/or for the antigens and/or fragments and/or antibodies coding nucleic acid sequences.

9. Kit for the diagnosis and/or therapy of Lyme Borreliosis, comprising glyceraldehyde-3-phosphate-dehydrogenase (GAPDH), oligopeptide-permease, oligopeptide ABC transporter periplasmic BP(oppA-2) (Bb), glycosyl transferase IgtD homolog, heat shock protein 90, VLSE fragment, (U76406) putative v1s rec. casette V1s6 Borrelia burgdorferi, flagellin protein Borrelia garinii, (AE001578), conserved hypothetical protein cp32-6 Borrelia burgdorferii, membrane assoc. protein p66 percursor Borrelia burgdoferi, oligopeptide ABC transporter periplasmic BP (oppA-4) (Bc), fructose-biphosphate aldose (fba) Borrelia burgdorferi, DNAK protein heat shock protein 70 Borrelia burgdorferi, orfE Borrelia burgdorferi, outer surface protein B precursor Borrelia burgdorferi, L-lactate dehydrogenase (ldh), P83/100 gene Borrelia burgdorferii, enolase 2-phosphoglycerate Borrelia burgdorferi, flagellin protein Borrelia garinii, hypothetical protein BBE28 Borrelia burgdorferi, DNA direct. RNA polymerase (rpoA) homolog, P66 protein (fragment), flagellin (fragment), DNA direct. RNA polymerase, integral outer membrane protein p66, pyruvate kinase (pyk) homolog, phosphoglycerate kinase (pgk) and/or BBU28760 NID and/or their fragments and/or antibodies and/or for the antigens and/or fragments and/or antibodies coding nucleic acid sequences.
